(19) United States
(12) Patent Application Publication
(10) Pub. No.: US 2017/0127981 A1
(43) Pub. Date: May 11, 2017

(54) HEMOGLOBIN DETECTION AND PHOTOPLETHYSMOGRAPHY USING SPECTRAL MODULATION

(71) Applicant: KONINKLIJKE PHILIPS N.V., EINDHOVEN (NL)

(72) Inventor: Olaf Thomas Johan Antonie Vermeulen, Eindhoven (NL)

(21) Appl. No.: 15/410,862

(22) Filed: Jan. 20, 2017

Related U.S. Application Data
(63) Continuation of application No. PCT/EP2015/066091, filed on Jul. 15, 2015.

(30) Foreign Application Priority Data
Jul. 30, 2014 (EP) 14179036.0

(51) Int. Cl. A61B 5/1455; A61B 5/00; A61B 5/024

(52) U.S. Cl. CPC A61B 5/1455; A61B 5/02416; A61B 5/14551; A61B 5/7257

(57) ABSTRACT

A hemoglobin detection apparatus comprises a spectrally tuneable emitter-detector unit, which is configured to emit or detect electromagnetic radiation spectrally selectively and periodically at different wavelengths covering a spectral modulation interval at a modulation frequency, and to provide a detector signal indicative of the detected electromagnetic radiation as a function of time. The apparatus further comprises a signal processing unit, which is configured to receive the detector signal and to provide an output signal, which is indicative of a contribution of at least one frequency component that forms a second or higher even harmonic of the modulation frequency to the detector signal. The hemoglobin detection apparatus can be used in photoplethysmography applications.

### CROSS REFERENCE TO RELATED APPLICATION

The present application is a Continuation of International Application No. PCT/EP2015/066091, filed Jul. 15, 2015, which claims priority to European Application No. 14179036.0, filed Jul. 30, 2014. These applications are incorporated herein by reference, for all purposes.

### TECHNICAL FIELD

The present embodiment relates to a hemoglobin detection apparatus and a hemoglobin detection method. It also relates to a photoplethysmography apparatus and a photoplethysmography method.

### BACKGROUND

Information about cardiovascular status, in particular blood parameters such as blood oxygen saturation, heart and respiratory rates can be acquired by photoplethysmography (PPG). PPG involves an optical acquisition of a plethysmogram, which is a measurement of a volumetric variation of tissue as a function of time.

Known PPG sensors are based on hemoglobin detection. U.S. Pat. No. 5,553,615A describes method and apparatus for a direct noninvasive prediction of hematocrit, i.e., percentage (in regards to volume) of red blood cells in in mammalian blood using PPG techniques and data processing. The method comprises selecting a plurality of wavelengths in the spectral range between 1150-2100 nanometer according to wavelength selection criteria. One or more of the following wavelength selection criteria must be satisfied for each of the different wavelengths used: for one wavelength the absorbance of water is at or near a measurable peak; for at least one wavelength the absorbance of oxyhemoglobin and deoxyhemoglobin are predictable and represent total hemoglobin content; for one wavelength the absorbance of water greatly exceeds the absorbance of all forms of hemoglobin; and for one wavelength the absorbance of all forms of hemoglobin greatly exceeds the absorbance of water. The method specified in claim 28 of U.S. Pat. No. 5,553,615A further comprises data processing steps to determine a hematocrit prediction from measured attenuated light intensity values at the different wavelengths.

Both, reflective and transmissive hemoglobin detection techniques are well known. Conventional PPG sensors monitor a perfusion of blood to the dermis and subcutaneous tissue of the skin through an absorption measurement at a specific wavelength. In conventional hemoglobin detection techniques the output signal comprises, in addition to a desired signal contribution from electromagnetic radiation transmitted through blood, a far greater signal contribution originating from transmission through and backscattering by other species, such as tissue, and by blood sloshing, i.e., venous blood movement. Low venous pressure blood “sloshes” with back and forth movement which is seen when an individual is physically active. This local perturbation of venous blood adds to the AC component of the detector signal.

In WO 2007/140422 A2, methods and systems for calculating tissue oxygenation, e.g., oxygen saturation, in a target tissue are disclosed. In some embodiments, the methods include: (a) directing incident radiation to a target tissue and determining reflectance spectra of the target tissue by measuring intensities of reflected radiation from the target tissue at a plurality of radiation wavelengths; (b) correcting the measured intensities of the reflectance spectra to reduce contributions thereto from skin and fat layers through which the incident radiation propagates; (c) determining oxygen saturation in the target tissue based on the corrected reflectance spectra; and (d) outputting the determined value of oxygen saturation

### SUMMARY OF THE EMBODIMENT

It is an object of the present embodiment to provide a hemoglobin detection apparatus and method that allows obtaining a particularly high contribution of the desired signal and can be used in photoplethysmography.

According to a first aspect of the present embodiment, a hemoglobin detection apparatus is provided. The hemoglobin detection apparatus comprises

a spectrally tuneable emitter-detector unit, which is configured to emit or detect electromagnetic radiation spectrally selectively and periodically at different wavelengths covering a spectral modulation interval at a modulation frequency, and to provide a detector signal indicative of the detected electromagnetic radiation as a function of time; and

a signal processing unit, which is configured to receive the detector signal and to provide an output signal, which is indicative of a contribution of at least one frequency component that forms a second or higher even harmonic of the modulation frequency to the detector signal.

The hemoglobin detection apparatus of the first aspect of the present embodiment allows obtaining an advantageously high contribution of the desired response of hemoglobin to the electromagnetic radiation provided by the emitter-detector unit to the output signal.

A periodic spectrally modulated emission or detection across a suitably selected spectral modulation interval introduces a desired contribution by hemoglobin to the detector signal at a frequency corresponding to a second or higher even harmonic of the modulation frequency. This desired contribution is identifiable and thus separable from other unwanted signal contributions to the detector signal by way of its frequency, which is an even harmonic of the modulation frequency. Therefore, the hemoglobin transmittance is determined independently from the transmittance contributions of other species to the detector signal, which provide contributions to the detector signal having uneven harmonics. The hemoglobin detection apparatus therefore allows an optical detection of hemoglobin and, thus, blood with a high rejection of interfering other species.

Other species are, in particular, epidermis, dermis and subcutaneous tissue including fat, of a living being, in particular a mammal, in particular a human being. Depending on the spectral range used for operation, water may or may not belong to the other species to be considered. For water exhibits spectral features in its transmittance spectrum that could disturb the measurement in particular in the infrared spectral region, but much less so in the visible spectral region.

The wavelength modulation used generates an amplitude modulation in the detector signal. This conversion thus also modulates the measurand. This has the advantage that the detector signal is transferred from a lower frequency to a higher frequency which separates the desired signal contribution from unwanted contributions generated by motion artefacts. As is well known, the motion of a person tested generates unwanted signal contributions, which can now be seperated from the desired signal contributions.

Another advantage is that the sensor is self-calibrating: the amplitude of the detector signal at the modulation frequency and its harmonics provide a measure for the amplitude, in other words, the attenuation by the tested sample, while the modulation contrast at the modulation frequency and its harmonics provide a measure for absorption by hemoglobin or other species.

The term wavelength is only used here as a common term for a reference to a spectral position for emission or detection of electromagnetic radiation. Any quantity describing the spectral position of emission or detection of electromagnetic radiation can be used instead, such as, e.g., an energy of the electromagnetic radiation provided in units of eV or wavenumbers. When referring to “different” wavelengths, no restriction regarding spectral width is intended that would restrict the spectrum of the emission or detection at any point in time to a single wavelength. As is well known, common sources of electromagnetic radiation provide emission having a certain spectral width. Also a spectrally selective detection allows a certain spectral width of electromagnetic radiation at each spectral position. In this regard, any spectral width of emission and detection providing a suitable spectral resolution for detecting a significant contribution by hemoglobin to the second or higher even harmonic of the modulation frequency to the detector signal is sufficient.

In the following, embodiments of the hemoglobin detection apparatus will be described.

The hemoglobin detection apparatus preferably employs a periodic spectral tuning of the emitter-detector unit over a spectral modulation interval, in which hemoglobin exhibits a particular spectral feature in its transmittance spectrum. This spectral feature is a nonlinear spectral dependence that can be decomposed with a significant contribution provided by at least one even function. As is well known, an even function f(x) has the property f(x)=f(−x). In the same spectral modulation interval, the same quantity indicative of the respective transmittance of other species to be exposed to the electromagnetic radiation emitted and detected, plotted as a function of wavelength, must not exhibit a nonlinear spectral dependence that can be decomposed with a significant contribution of at least one even function. As will be shown in the present disclosure, these criteria are fulfilled in various intervals of the spectrum of electromagnetic radiation. In such spectral intervals, which are suitable for forming the spectral modulation interval, the hemoglobin detection apparatus achieves a particularly high degree of separation of signal contributions from hemoglobin and other species in the frequency domain.

Any measure that allows sensing a spectral feature with a nonlinear spectral dependence that can be decomposed with a significant contribution by at least one even function in a response of hemoglobin and of the other species exposed to electromagnetic radiation can be used for the hemoglobin detection in accordance with the present embodiment. Typically, the response includes absorption and or scattering of the electromagnetic radiation. A suitable measure indicative of the absorbance and scattering characteristics of a sample is for instance its transmittance. The transmittance of a sample exposed to electromagnetic radiation of a given wavelength is a measure for the intensity fraction of the electromagnetic radiation that passes through the species and is therefore not subject to absorption, nor to scattering of the electromagnetic radiation by the sample. Different absorption mechanisms as well as elastic and inelastic physical scattering mechanisms of electromagnetic radiation are known per se to a person of ordinary skill in the art, and the spectral dependence of the relevant species are either available or can be determined by measurement.

A significant contribution of an even function to a decomposition to the spectral dependence of the hemoglobin transmittance in the spectral modulation interval generates and thus corresponds to a significant contribution to the detector signal by hemoglobin with at least one even harmonic of the modulation frequency.

A quantification of a contribution of a given even harmonic required for achieving significance is determined by the person of ordinary skill in the art in routine work. In particular, the selection of the spectral modulation interval influences the criterion for a significant contribution. Where only hemoglobin (and no other species) provides a signal at a selected even harmonic of the modulation frequency in the Fourier spectrum of the detector signal, a significant contribution to the detector signal is achieved with a minimum amplitude required to detect the presence of the respective harmonic in the detector signal. On the other hand, when using a spectral modulation interval, in which this situation cannot be fully achieved and other species are known to contribute to the detector signal at the selected even harmonic, other criteria for significance may apply. In particular, a the relative amplitude of the selected even harmonic in comparison with the relative amplitudes of other selected harmonics can be used to identify the significant contribution. In some embodiments, significance is given if the relative amplitude of the selected even harmonic is higher than that of all other harmonics of the modulation frequency in the Fourier spectrum of the detector signal.

In the above definition of the hemoglobin detection apparatus of the present embodiment, the phrase “plotted as a function of wavelength” is merely used to point to a spectral dependence of the transmittance.

The emitter-detector unit is configured to provide a spectral resolution that allows detecting the contribution of an even function to the spectral dependence of the transmittance of hemoglobin by emission and detection of electromagnetic radiation at different spectral positions in the spectral modulation interval.

In some embodiments, the particular spectral feature of a significant contribution by at least one even function can additionally be described as a change of sign of the slope of the quantity indicative of a transmittance of hemoglobin for electromagnetic radiation. In other words, the slope of the transmittance spectrum of hemoglobin changes from positive to negative or from negative to positive within the spectral modulation interval. In the spectral modulation interval the same quantity indicative of the transmittance of other species exposed to the emitted electromagnetic radiation in operation of the apparatus does not exhibit a change of sign of the respective slope.

Both directions of change of the sign of the slope are suitable for a selection of the spectral modulation interval. The spectral feature used by the hemoglobin apparatus thus is in some embodiments the occurrence of an extremum, i.e., maximum or minimum, of the transmittance within the spectral modulation interval under consideration.

Whether the spectral feature is a maximum or minimum also depends on the particular quantity measured as an indication of the transmittance of hemoglobin. For instance, while an absorbance of hemoglobin is indicative of the transmittance of hemoglobin, the absorbance and transmittance of a substance typically exhibit mutually complementary spectral features, meaning that at spectral positions, where the transmittance exhibits minima, the absorbance exhibits maxima. Any quantity indicative of the transmittance of hemoglobin in the spectral modulation interval can be used.

Some embodiments of the hemoglobin detection apparatus are configured to detect oxygenated hemoglobin, which is also referred to as oxyhemoglobin and is hemoglobin with bound oxygen. To this end, the emitter-detector unit is configured to emit or detect electromagnetic radiation spectrally selectively at different wavelengths covering the spectral modulation interval, in which the quantity indicative of a transmittance of oxyhemoglobin, plotted as a function of wavelength, exhibits the significant contribution of an even function to the spectral dependence, for instance in the form of slopes of opposite signs, and in which spectral modulation interval the quantity indicative of the respective transmittance of other species, such as epidermis, dermis and hypodermis, plotted as a function of wavelength, does not exhibit a significant contribution of an even function to the spectral dependence, such as the mentioned change of sign of the respective slope.

Other embodiments are configured to additionally or alternatively detect deoxyhemoglobin, which is hemoglobin without bound oxygen. To this end, the emitter-detector unit is additionally or alternatively configured to emit or detect electromagnetic radiation spectrally selectively at different wavelengths covering a spectral modulation interval, in which a quantity indicative of a transmittance of deoxyhemoglobin, plotted as a function of wavelength, exhibits the significant contribution of an even function to the spectral dependence, for instance in the form of slopes of opposite signs, and in which spectral modulation interval the quantity indicative of the respective transmittance of other species, such as for example epidermis, dermis and hypodermis, plotted as a function of wavelength, does not exhibit a significant contribution of an even function to the spectral dependence, such as the mentioned change of sign of the respective slope.

In embodiments suitable for the detection of oxygenated hemoglobin but not of deoxygenated hemoglobin, the spectral modulation interval preferably includes wavelength of 416 nm, 516 nm, 540 nm or 576 nm, where suitable spectral features of only oxygenated hemoglobin are available.

In embodiments suitable for the detection of deoxygenated hemoglobin, but not of oxygenated hemoglobin, the spectral modulation interval preferably includes the wavelength of 434 nm, 736 nm or 758 nm, where suitable spectral features of only deoxygenated hemoglobin are available.

In some embodiments, the quantity indicative of the transmittance is an intensity of electromagnetic radiation transmitted through a sample to be measured or electromagnetic radiation backscattered from regions of a sample to be measured. In the first case, the sample to be measured is for instance an ear lobe. In the second case, the sample to be measured is for instance a finger or wrist. A combination of both quantities may be used in other embodiments.

In one embodiment of the hemoglobin detection apparatus the spectral modulation interval, in which the quantity indicative of a transmittance of hemoglobin exhibits a significant contribution of an even function to the spectral dependence, such as the mentioned slopes of opposite signs, comprises a wavelength, at which oxygenated hemoglobin exhibits a local peak or a local minimum of absorbance, which may in some variants take the form of an absorption peak that has a resonance line shape.

Different suitable central wavelengths of spectral modulation intervals in which the electromagnetic radiation is provided are at spectrally narrow absorbance extrema of oxyhemoglobin near 416 nm, 512 nm, 542 nm, 560 nm, 576 nm. The spectral modulation interval must be small enough to avoid inclusion of undesired spectral features in the spectral modulation interval, which could introduce a perturbation of the desired signal. On the other hand, the spectral modulation interval must be wide enough to allow a reliable detection of the change of sign of the slope of the transmittance quantity.

Another suitable type of central wavelength of a suitable spectral modulation interval is an absorbance minimum, for instance a minimum between two absorbance peaks. In one such embodiment, the central wavelength is 684 nm. Oxyhemoglobin exhibits a broad minimum of absorbance in this spectral region. Therefore, the spectral modulation interval is suitably selected with a larger width to allow a reliable detection of the change of sign of the slope of the transmittance quantity.

Embodiments of the homoglobin detection apparatus have an emitter-detector unit with a detector unit that comprises a solid-state detector device, which is configured to provide the detector signal as an electrical signal in correspondence to an intensity of detected electromagnetic radiation in the spectral modulation interval. Suitable detector device are a photodiode or another device suitable to generate an electrical signal in response to irradiation with electromagnetic radiation in the spectral modulation interval.

The harmonic component most suitable for providing as an output depends on the peculiarities of the spectral feature used for the detection.

In some embodiments the signal processing unit is configured to provide as the output signal the contribution of only the second harmonic of the modulation frequency to the detector signal. This embodiment is particularly simple and allows a reliable detection of a single maximum or minimum of the transmittance of hemoglobin in the spectral modulation interval. In case the spectral modulation interval comprises three local extrema, i.e., 3 changes of sign of the slope sign, a dominant fourth harmonic is generated and can be used for hemoglobin detection.

One embodiment comprises a spectral alignment unit, which is configured to control the modulation control unit in performing a spectral alignment process by testing different candidate spectral modulation intervals around a fixed central wavelength, the spectral modulation intervals having different upper or lower boundary wavelengths. Furthermore, the spectral alignment unit is preferably configured to determine from the respective detector signals received for the different candidate spectral modulation intervals an optimal spectral modulation interval, at which the contribution of the second or higher even harmonic of the modulation frequency to the detector signal is relatively the largest. It is preferably further configured to select the optimal spectral modulation interval as the spectral modulation interval to be used for regular operation for hemoglobin detection by the modulation control unit.

Different types of emitter-detector units can be used to implement different embodiments.

To achieve a good signal-noise-ratio, the emitter detector unit is preferably configured to provide or detect the electromagnetic radiation at at least three different wavelengths within the selected spectral modulation interval, such that the transmittance exhibits a particularly high contrast between a spectral position, where the transmittance assumes an extremum and spectral positions at higher and lower wavelengths in comparison with that of the extremum. For instance, considering a typical absorption feature with line shape corresponding to or resembling that of a single resonance peak, the three wavelengths may be suitably chosen at spectral positions a), b), and c), wherein the slope of the transmittance of hemoglobin is positive for spectral position a), negative for spectral position b), and zero or near zero for spectral position c), the latter spectral position corresponding to a wavelength between those of spectral positions a) and b). This way, the respective extremum forming a absorption feature of the hemoglobin transmittance spectrum with a substantial even functional contribution is tested on both edges (outer spectral positions) and at or near the peak of the absorption feature (central spectral position) during a given modulation period. When scanning the spectral modulation interval containing an extremum of transmittance this way, a high contrast ratio of transmittance can be achieved, resulting in a good signal-noise ratio of the output signal. The contrast ratio can be made particularly high by selecting wavelengths, which in comparison provide a particularly large difference in the respective transmittance values of hemoglobin. Since the transmittance spectra of all forms of hemoglobin, in particular oxyhemoglobin, deoxyhemoglobin and the dysfunctional hemoglobins carboxyhemoglobin (CoHb), methemoglobin (MetHb) and sulfhemoglobin (SulfHb) are per se known, a suitable wavelength selection is a design choice that can be made in the design phase of a particular embodiment of the hemoglobin detection apparatus.

However, it should be noted that it is not a requirement that the slopes at the outer spectral positions a) and b) used for measurement have opposite signs. It is only the selected spectral modulation interval as a whole that shall fulfill this requirement. As an example for the purpose of explanation of this point, a selected spectral modulation interval covered by three different selected wavelengths may fulfill the requirement of a change of sign of the slope of the transmittance of a hemoglobin species, but all three spectral positions (i.e., three different wavelengths) used for measurement exhibit a respective slope of zero, or a positive slope for both outer spectral positions measured, or even a positive or negative slope for all three spectral positions used in the transmittance spectrum of hemoglobin. It is only important that the selected spectral modulation interval includes a spectral feature that is unique for hemoglobin in comparison with spectral features of the other species, in particular epidermis, dermis and hypodermis, which are exposed to the electromagnetic radiation which is emitted and detected in the hemoglobin detection measurement.

In some embodiments, the emitter-detector unit comprises a spectrally tuneable emitter unit, which is configured to selectively provide the electromagnetic radiation at different wavelengths. In such embodiments, a detector unit is preferably used, which is configured to provide a detector signal that is indicative of an amount of electromagnetic radiation emitted by the emitter unit and scattered by blood and the other species, such as epidermis, dermis and subcutaneous tissue of a subject, as a function of time.

Such embodiments with a spectrally tuneable emitter unit can be implemented in different ways. In one such embodiment of the hemoglobin detection apparatus the emitter unit comprises at least one tuneable solid-state emitter. Examples are a tuneable LED, or a tuneable OLED, or a tuneable laser diode.

In another embodiment comprising a spectrally tuneable emitter unit, the emitter unit comprises a plurality of different solid-state emitters, each providing one fixed wavelength within the spectral modulation interval, and to activate a respective one of the different solid-state light emitters at a respective phase of the modulation period.

Another variant of a hemoglobin detection apparatus with a spectrally tuneable emitter unit comprises a plurality of different solid-state emitters with mutually overlapping emission spectra. In this variant a respective relative intensity of each of the different solid-state emitters is varied at a respective phase of the modulation period. This may for example be achieved with an intensity control signal that in one embodiment is a vector signal that comprises a plurality of parallel signals, one for each emitter.

Yet another variant of a spectrally tuneable emitter unit comprises a tuneable optical filter, which is configured to transmit the electromagnetic radiation at one of a plurality of different selectable wavelengths across the spectral modulation interval. In this variant, the emitter unit preferably comprises an emitter, which is configured to provide the electromagnetic radiation with a fixed emission spectrum that covers the spectral modulation interval.

In another group of embodiments of the hemoglobin detection apparatus, the emitter-detector unit comprises a spectrally tuneable detector unit, which is configured to selectively detect the electromagnetic radiation at different wavelengths and to provide a detector signal that is indicative of an amount of the spectrally selected electromagnetic radiation emitted by the emitter unit and scattered blood and other species of a subject, as a function of time.

In one embodiment of this group the detector unit comprises a tuneable optical filter, which is configured to transmit the electromagnetic radiation at one of a plurality of different selectable wavelengths across the spectral modulation interval, for example in dependence on a received tuning control signal. Different variants of this embodiment use different tuneable filter alternatives, such as a grating or prism monochromator, a tuneable liquid crystal optical filter, or a suitably selected timely sequence of optical band pass filters.

In some variants this group of embodiments of the hemoglobin detection apparatus the emitter unit comprises an emitter, which is configured to provide the electromagnetic radiation with a fixed emission spectrum that fully covers the spectral modulation interval.

A combination of a spectrally tuneable emitter unit with a spectrally tuneable detector unit is considered and may be advantageous for some embodiments, for instance to achieve a particularly high suppression of unwanted signal contributions to the detector signal.

Some embodiments further comprise a modulation control unit, which is configured to provide a tuning control signal, which is periodic at a modulation frequency fₘ for driving a spectrally modulated emission or detection of electromagnetic radiation by the emitter-detector unit that covers the spectral modulation interval during a respective modulation period.

Frequency-specific signal processing can be performed using a signal processing unit that comprises a lock-in amplifier, which receives the tuning control signal and the detector signal. In another embodiment, a synchronous detector is used instead of a lock-in amplifier. In yet another embodiment, a band-pass filter centered around a predetermined even harmonic of the modulation frequency is used. Another embodiment has a signal processing unit comprising a combination of a band-pass filter with a lock-in amplifier of a synchronous detector.

It is noted that the components used in the emitter unit and in the detector unit typically have spectral characteristics that need to be taken into account in the design to avoid incorrect detection results. For instance, a detector such as a photodiode with a certain sensitivity peak around a transmittance peak of the measurand could incorrectly suggest a detection of hemoglobin even if it was not present. To avoid the necessity of a correction unit, such issues can be handled by proper selection of suitable emitter and detector components with a view to their spectral characteristics in the selected spectral modulation interval. However, a certain slope in emission intensity and detector sensitivity may not be avoidable. Therefore, where required, any of the embodiments of the present disclosure can be extended with a correction unit, which is configured to compensate spectral characteristics of the components used for emission or detection of the electromagnetic radiation. On the detection side, the correction unit may be implemented as one of the initial stages of the signal processing unit. The correction unit corrects signal distortions attributable to the known spectral dependence of the emission intensity and of the detection sensitivity. Another possibility is a spectral pre-equalization stage on the emission side, for example as a part of the tuning control unit, for controlling an intensity of the electromagnetic radiation provided by the emitter unit at the different wavelengths used in a modulation period. This way, a compensation for both emitter and detector characteristics can be achieved. The pre-equalization stage can be based on a priori setting of intensities, or use a feedback loop with an additional detector of the same kind as that used for the actual hemoglobin detection measurement.

In another embodiment, the correction unit is additionally or alternatively configured to apply a signal correction to at least one of the second or higher even harmonic signal contributions, which contain the actual desired signal, using that component of the detector signal which has the modulation frequency, and which can thus be considered as the “carrier” signal. This embodiment is based on the recognition that both the “carrier” signal and the higher-frequency “sideband” signals, which contain the desired signal, may be influenced by distortions such as motion artefacts in a similar way. The envelope amplitude as a function of time detected at the modulation frequency can thus provide a basis for deriving a signal correction to be applied to the “sideband” signal. The signal correction is in one embodiment the envelope of the “carrier” signal modified by a scaling factor, which can be suitably selected by routine operation.

An advantageous application case of the hemoglobin detection apparatus is a photoplethysmography apparatus, which comprises the hemoglobin detection apparatus according to the first aspect of the embodiment or one of its embodiments, and which further comprises a PPG evaluation unit, which receives the output signal and is configured to determine from the output signal and provide cardiovascular status information, in particular blood oxygen saturation, heart rate, and respiratory rate

According to a second aspect of the embodiment, a hemoglobin detection method is provided. The method comprises

periodically providing, at a modulation frequency, a spectrally selective emission and detection of electromagnetic radiation at different wavelengths that during a respective modulation period cover a spectral modulation interval;

providing a detector signal indicative of the detected electromagnetic radiation as a function of time; and

processing the detector signal and providing an output signal, which is indicative of a contribution of at least one frequency component that forms a second or higher even harmonic of the modulation frequency to the detector signal.

The method of the second aspect of the embodiment shares the advantages of the hemoglobin detection apparatus of the first aspect of the embodiment.

One embodiment of the method comprises

providing a spectrally tuneable emitter-detector unit;

providing a tuning control signal to the emitter-detector unit, which tuning control signal is periodic at a modulation frequency, thus driving a periodic spectrally selective emission and detection of electromagnetic radiation at different wavelengths that cover a spectral modulation interval during a respective modulation period, in which a quantity indicative of a transmittance of hemoglobin, plotted as a function of wavelength, exhibits a nonlinear spectral dependence that can be decomposed with a significant contribution by at least one even function, and in which spectral modulation interval the quantity indicative of the respective transmittance of other species to be exposed to the electromagnetic radiation emitted and detected, plotted as a function of wavelength, do not exhibit a nonlinear spectral dependence that can be decomposed with a significant contribution of at least one even function;

providing a detector signal indicative of the detected electromagnetic radiation as a function of time; and

processing the detector signal and providing an output signal, which is indicative of a contribution of at least one frequency component that forms a second or higher even harmonic of the modulation frequency to the detector signal.

An advantageous application case of the hemoglobin detection method of the second aspect of the embodiment is a photoplethysmography method, comprising a hemoglobin detection method according to the second aspect of the embodiment or one of its embodiments, and further comprising determining from the output signal and providing cardiovascular status information.

It shall be understood that the hemoglobin detection apparatus of the first aspect of the embodiment, as also defined in claim 1, the hemoglobin detection method of the second aspect of the embodiment or claim 14 have similar and/or identical embodiments, in particular, as defined in the dependent claims 2 to 13.

### BRIEF DESCRIPTION OF THE DRAWINGS

In the following drawings

FIG. 1 shows a block diagram of an embodiment of a hemoglobin detection apparatus and of a PPG apparatus;

FIG. 2 shows a block diagram of another embodiment of a hemoglobin detection apparatus and a PPG apparatus;

FIG. 3 is an illustration of a working principle of hemoglobin detection apparatus and PPG apparatus according to the present embodiment;

FIGS. 4 and 5 each show a schematic block diagram of emitter units of different embodiments of a hemoglobin detection apparatus according to the present embodiment;

FIG. 6 is a diagram showing transmittance properties of hemoglobin and oxygenated hemoglobin in a spectral range between 200 and 1000 nanometer for illustrating suitable spectral ranges for implementation of a hemoglobin detection apparatus of the present embodiment;

FIG. 7 compares absorption properties of blood with those of melanosome, epidermis and skin, for identifying suitable spectral regions for implementation of hemoglobin detection and PPG according the present embodiment;

FIG. 8 is a section of the spectrum of FIG. 6 illustrating a suitable spectral modulation interval for use in an embodiment of the hemoglobin detection apparatus and of the PPG apparatus;

FIGS. 9 to 13 show the same section of the spectrum of FIG. 6 as FIG. 8 for illustrating different spectral modulation intervals for implementation of embodiments of a hemoglobin detection apparatus;

FIGS. 14 to 17 show different alternative sets of suitable wavelengths in the same given spectral modulation interval in the implementation of different embodiments of a hemoglobin detection apparatus;

FIGS. 18 to 21 show examples of a frequency distribution of the detector signal for different spectral modulation intervals in the implementation of different embodiments of a hemoglobin detection apparatus;

FIG. 22 is a flow diagram showing an embodiment of a hemoglobin detection method; and

FIG. 23 is a flow diagram of an embodiment of a photoplethysmography method of an embodiment of a photoplethysmography method.

### DETAILED DESCRIPTION OF EMBODIMENTS

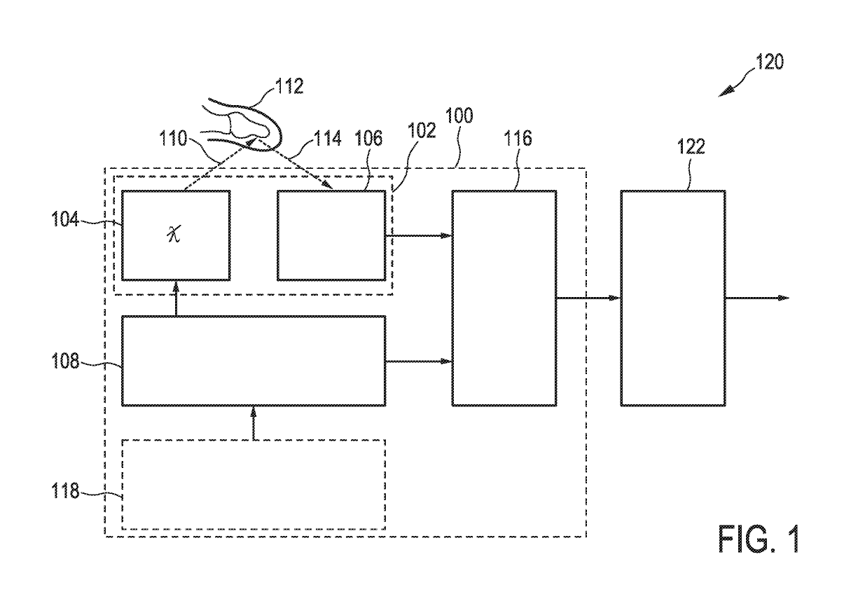

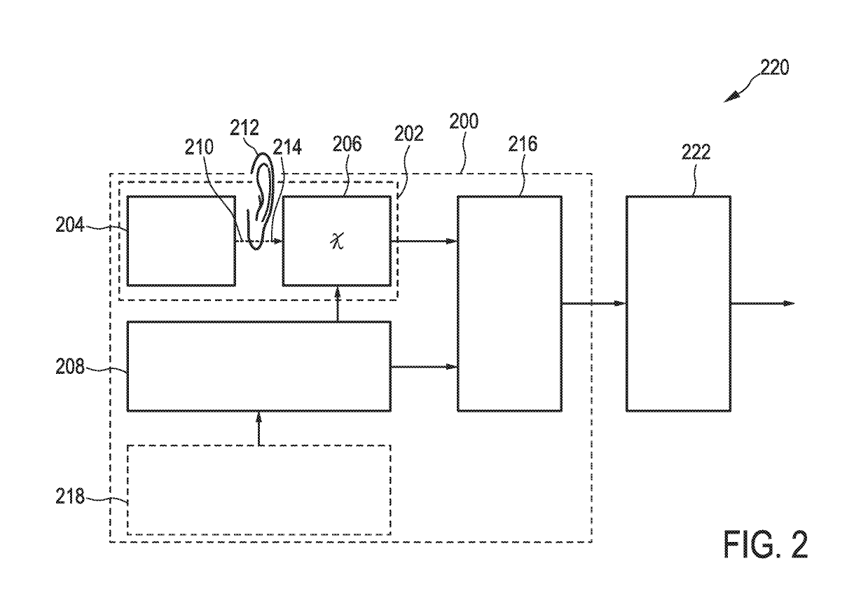

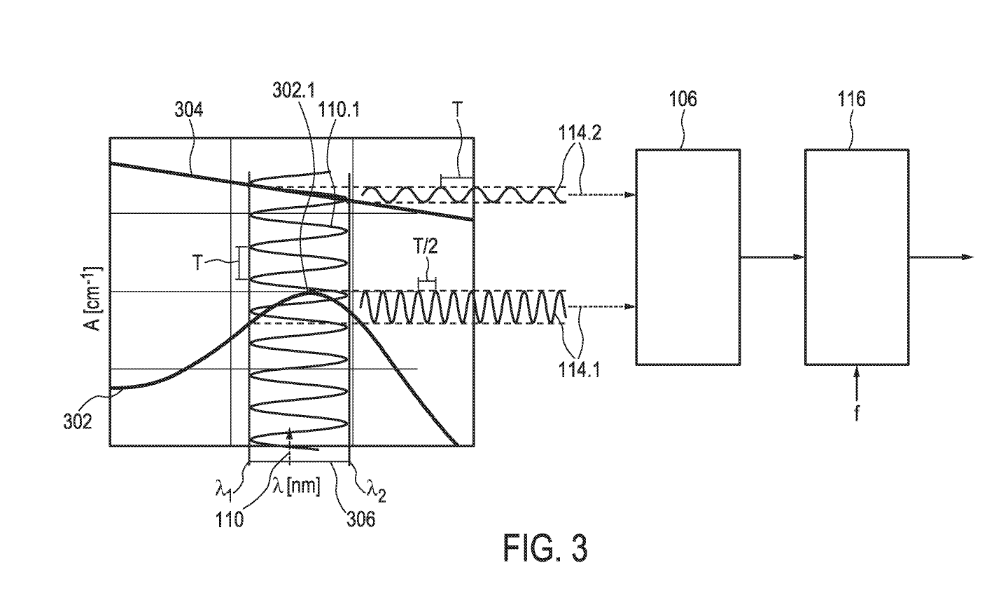

The following description of the FIGS. 1 to 3 first turns to the structure of embodiments of hemoglobin detection apparatus and PPG apparatus according to the present disclosure, as shown in FIGS. 1 and 2. Subsequently, the function of these embodiments will be described with reference to an illustration of a working principle, which is common to both embodiments, given in FIG. 3.

FIG. 1 shows a block diagram of an embodiment of a hemoglobin detection apparatus 100 and of a PPG apparatus 120. The hemoglobin detection apparatus is fully comprised by the PPG apparatus 120.

The hemoglobin detection apparatus 100 comprises a spectrally tuneable emitter-detector unit 102, which comprises a spectrally tuneable emitter unit 104 and a detector unit 106.

The spectrally tuneable emitter unit 104 receives input from a modulation control unit 108. More specifically, the modulation control unit 108 generates and provides to the emitter unit 104 a tuning control signal. The tuning control signal is periodic at a modulation frequency. In one embodiment, the modulation control unit 108 comprises an oscillator and a driver for driving the emission. The driver is in a variant comprised by the emitter unit 104 and not by the modulation control unit 108.

In the present embodiment, the periodic tuning control signal provided to the emitter unit 104 serves for driving a spectrally modulated emission of electromagnetic radiation that covers a spectral modulation interval during a respective modulation period. In response to the received tuning control signal, the emitter unit 104 emits electromagnetic radiation spectrally selectively at different wavelengths, as determined by the tuning control signal. However, the provision of a tuning control signal generated by a modulation control unit is not a requirement. Other solutions for providing the desired periodic spectrally modulated emission can be employed.

The different wavelengths cover the spectral modulation interval. The spectral modulation interval is selected according to the following criteria:

a) A quantity indicative of a transmittance of hemoglobin, plotted as a function of wavelengths, exhibits slopes of opposite signs within this spectral modulation interval; and

b) Furthermore, the quantity indicative of the respective transmittance of epidermis, dermis and hypodermis, plotted as a function of wavelength, does not exhibit a change of sign of the respective slope within this spectral modulation interval.

The electromagnetic radiation emitted in the spectral modulation interval is indicated by a dashed arrow 110. The hemoglobin detection apparatus 100 works in a back-scattering mode, which as such is known in the art. Electromagnetic radiation 110 is emitted and transmitted through the epidermis, dermis to reach subcutaneous tissue, i.e. the hypodermis, of a finger 112 in order to reach blood vessels containing hemoglobin. Hemoglobin is normally not present in the epidermis, dermis and those parts of the hypodermis that are different from blood vessels. The fraction of the impinging electromagnetic radiation, which is back-scattered by the irradiated tissue of the finger 112 and reaches the detector unit after transmission through the mentioned tissue regions of the finger 112 is indicated in FIG. 1 by a dashed arrow carrying the reference label 114. The back-scattered electromagnetic radiation is detected by the detector unit 106. The detector unit 106 is sensitive, i.e., configured to detect received electromagnetic radiation in the whole spectrum covered by the selected spectral modulation interval. The detector unit 106 comprises, e.g., a photodiode or a phototransistor. The detector is in one variant of the present embodiment provided with a band pass filter that allows light to pass substantially only in the selected spectral modulation interval in order to reject ambient light. But this is not a requirement.

In the present embodiment, therefore, the spectral selectivity and tuneability allowing a determination of the transmittance of the mentioned tissue regions of the finger 112 as a function of wavelength within the spectral modulation interval is provided by the emitter unit 104 of the hemoglobin apparatus 100. Therefore, it is not required to provide the tuning control signal to the detector unit 106.

The hemoglobin detection apparatus 100 further comprises a signal processing unit 116, which is configured to receive the detector signal provided by the detector unit 106. Furthermore, the signal processing unit 116 is configured to provide an output signal, which is indicative of a contribution of at least one frequency component that forms a second or higher harmonic of the modulation frequency to the detector signal. Different ways of implementation of the signal processing unit 116 will be described in the context of the description of FIG. 3.

In operation, the wavelength modulated light is directed towards the skin and its transmission or reflection is detected by the detector unit 106. The detector signal, for instance a detected photocurrent, is processed by the signal-processing unit. The processing for instance comprises amplification and conversion to the digital domain with an ADC. A digital lock-in amplifier, which receives its reference frequencies from the modulation control unit that also controls the modulation of the tuneable emitter unit 104, separates the desired signals from undesired signals. The lock-in detector can of course also be implemented in the analogue domain.

Details of the operation underlying the different functional units of the hemoglobin detection apparatus 100 will be described further below with reference to FIG. 3.

A variant of the hemoglobin detection apparatus 100 additionally comprises a spectral alignment unit 118 which in FIG. 1 is represented by a box with a dashed outline to indicate that this unit is optional. The functionality of the spectral alignment unit will be described further below after explanation of the operation principle of the hemoglobin detection apparatus with reference to FIG. 3.

In another embodiment, which is also illustrated in FIG. 1, the hemoglobin detection apparatus 100 may form an integrated component of a PPG apparatus, which is referred to under the reference label 120. In order to determine PPG information from the output provided by the signal processing unit 116, the hemoglobin detection apparatus 100 is extended to form a PPG apparatus 120 by further providing a PPG evaluation unit 122, which receives the output signal of the hemoglobin detection apparatus 100 provided by the signal processing unit 116 and which is configured to determine from the output signal and provide at its output cardiovascular status information, such as heart rate information, oxygen saturation information or other information.

Before turning to a detailed description of the operation of the hemoglobin detection apparatus 100 and of the PPG apparatus 120 illustrated in FIG. 1, an alternative embodiment of a hemoglobin apparatus 200 and a PPG apparatus 220 will be described with reference to FIG. 2.

FIG. 2 shows a block diagram of another embodiment of a hemoglobin detection apparatus and a PPG apparatus. The hemoglobin detection apparatus 200 is similar to that of FIG. 1. Therefore, the following description focuses on the differences between the embodiments of FIGS. 1 and 2.

In the hemoglobin detection apparatus 200, the spectral selectivity and tuneability allowing a determination of the transmittance of tissue regions including blood vessels as a function of wavelength within the spectral modulation interval is provided by the detector unit 206 of the hemoglobin apparatus 200.

In contrast to the hemoglobin detection apparatus 100 of FIG. 1, therefore, the hemoglobin detection apparatus 200 of FIG. 2 has an emitter unit, which is configured to provide the electromagnetic radiation 210 with a fixed emission spectrum that covers a predetermined spectral modulation interval. Furthermore, the detector unit 206 of the emitter-detector unit 202 of the hemoglobin detection apparatus 200 is spectrally tuneable. In other words, it is configured to provide spectral selectively and tuneability in the detection of electromagnetic radiation at different wavelengths within the selected spectral modulation interval in dependance on the tuning control signal.

A further difference to the embodiment of FIG. 1 is that the present hemoglobin detection apparatus is configured to operate in a transmission mode, in which electromagnetic radiation is transmitted through tissue, which by way of a non-restrictive example is an ear lobe of an ear 212. In other words, the spectrally tuneable detector unit is configured to provide a detector signal that is indicative of an amount of the electromagnetic radiation emitted by the emitter unit and scattered in transmission mode by the dermis, subcutaneous tissue and blood vessels of the ear lobe of the ear 212 and then spectrally selected (e.g., filtered) by the detector unit, as a function of time.

It is noted that the use of a transmission or backscattering mode is a product design choice that can be made independently from the technique used for providing spectral selectivity and tuneability. Variants of the hemoglobin detection apparatus 100 and 200 are configured for operation using the respective other of the operational modes illustrated, i.e., transmission or back-scattering mode.

To achieve spectral selection and spectral tuning in the detector unit 206, the modulation control unit 208 of hemoglobin detection apparatus 200 of FIG. 2 provides the tuning control signal to the detector unit 206. It is not necessary to provide the tuning control signal to the emitter unit 204. The spectral selection and spectral tuning can be achieved by a spectrally tuneable filter, by a grating monochromator or any other technique known per se.

The signal processing performed by the signal processing unit 216 can be identical to that of the embodiment of FIG. 1.

As in the embodiment of FIG. 1, there is also the option of providing a spectral alignment unit 218.

Furthermore, in one embodiment the hemoglobin detection apparatus 200 forms an integral part of a PPG apparatus 220, which is also illustrated in FIG. 2 and further comprises a PPG evaluation unit 222 for determining cardiovascular status information from the output provided by the hemoglobin detection apparatus 200.

FIG. 3 is an illustration of a working principle of hemoglobin detection apparatus and PPG apparatus according to the present disclosure, and, in particular, according to the embodiments of FIGS. 1 and 2. FIG. 3 shows a diagram representing a section of an absorption spectrum of oxygenated hemoglobin in arbitrary linear units, plotted as a function of the wavelength λ in nanometer. The absorption spectrum of oxygenated hemoglobin is shown under reference label 302. Also shown in the diagram of FIG. 3 is an absorption spectrum 304 of melanin. Melanin forms a main interfering species in a hemoglobin detection measurement an thus also in photoplethysmography. Melanin is shown as an example of such interfering species, which includes epidermis, dermis and any species, which is comprised by the hypodermis and different from hemoglobin.

The spectral modulation interval 306 is selected to cover a spectral region, in which the slope of the hemoglobin absorption spectrum as a function of wavelength assumes opposite signs. In the selected spectral modulation interval 306, the absorption spectrum of oxygenated hemoglobin exhibits a resonance absorption feature with a maximum 302.1. Thus, in the present example, taking the spectral position of a maximum 302.1 of the resonance absorption feature as a reference wavelength, the slope of the absorption as a function of wavelength is positive at smaller wavelengths, and the slope of the absorption spectrum is negative at larger wavelengths. In contrast, the absorption spectrum of melanin is continuously decreasing in this spectral modulation interval without any change of sign in its slope. Other species are not shown, but are known and can be assumed to exhibit a similar behaviour as melanin.

In many embodiments, the quantity actually measured is not the absorption coefficient, but another quantity indicative of the transmittance of a sample. As is well known, the absorption coefficient in cm⁻¹ is complementary to the transmittance of a sample measured. The higher the absorption, the smaller is the transmittance. Thus, the transmittance of the measured sample will assume a minimum at the spectral position of the maximum 302.1 shown in FIG. 3, and the slopes of the transmittance features towards smaller and larger wavelengths are opposite in the transmittance spectrum in comparison with the absorption spectrum. However, this does not matter. The occurrence of a change of the sign of the slope in the measured quantity matters.

In the hemoglobin detection apparatus 100 of FIG. 1, the wavelength of the emitted electromagnetic radiation 110, which covers the spectral modulation interval 306, is provided by the emitter unit 104 and is modulated in accordance with the tuning control signal. In the illustrative example represented in FIG. 3, the provided wavelength oscillates periodically and continuously in the spectral limits given by wavelengths λ₁ and λ₂ forming the lower and upper limits of the spectral modulation interval 306. This is illustrated by an oscillating waveform 110.1 in FIG. 3, which is to be understood as a representation of the wavelength (along the abscissa) as a function of time (in the direction of the ordinate) having a modulation period T which corresponds to a modulation frequency f=1/T. By providing an oscillating spectral modulation of the electromagnetic radiation 110, the contribution 114.2 of melanin to the transmitted electromagnetic radiation 114 and thus to the detector signal is in the frequency domain mostly concentrated at the modulation frequency of the incoming electromagnetic radiation, due to the almost linear absorption spectrum of melanin in the spectral modulation interval 306. In contrast, the contribution 114.1 of oxygenated hemoglobin to the transmitted electromagnetic radiation 114, according to its resonance absorption feature at the spectral position 302.1, exhibits a modulation with a predominant contribution of a modulation period T/2 that in the frequency domain corresponds to the second harmonic frequency 2f of the modulation frequency f of the incoming electromagnetic radiation.

This is achieved as follows: during a single period of the oscillation of the wavelength across the spectral modulation interval 306, the resonance peak of absorption 302.1 will be scanned twice, thus adding a signal contribution having two periods of oscillation within a single modulation period to the detector signal. Therefore, the contribution of oxygenated hemoglobin to the overall detector signal is made identifiable by its modulation frequency according to a second and other even harmonic of the modulation frequency of the tuning control signal, while the contribution of melanin (and other species) is characterized by its modulation frequency identical to the modulation frequency and other odd harmonics thereof. Depending on the specific spectral dependence of the transmittance, small contributions harmonics higher than the second harmonic may be provided by all species including hemoglobin. Thus, by separating the different frequency contributions to the detector signal in the signal processing unit 116, the output of the signal processing unit selectively provides the contribution of the frequency component 2f generated substantially alone by oxygenated hemoglobin.

The signal contribution of hemoglobin to the detector signal can thus be identified by its modulation frequency and can be separated from other frequency components of the detector signal. The separation may be achieved in the signal processing unit 116 by synchronous detection or by employing a lock-in technique. Different embodiments employ either a digital lock-in technique or an analog lock-in technique. In any case, the signal processing unit 116, 216 suitably also receives the tuning control signal provided by the modulation control unit, as indicated by corresponding arrows between the modulation control unit 108 and the signal processing unit 116, and by the modulation control unit 208 and the signal processing unit 216. The modulation frequency is chosen such that a clear separation with a known maximum frequency of signal contributions provided by motion artefacts is achieved.

The implementation of hemoglobin detection according to embodiments of the present disclosure has been described in the previous paragraphs for the case of embodiment of the hemoglobin detection apparatus 100 of FIG. 1, which has a spectrally modulated tuneable emitter 104. The operation principle is the same for the case of a spectrally modulated tuneable detector unit 206, as in the hemoglobin detection apparatus 200 of FIG. 2. For in both cases, the transmittance is detected spectrally selectively in a periodic manner under control of the modulation control unit 108, 208. Thus, the explanations given above for the embodiment of FIG. 1 also apply to the embodiment of FIG. 2.

The technique described by way of the exemplary embodiments of FIGS. 1 and 2 allows a detection of hemoglobin, and therefore of blood with a high rejection of interfering species. The employed spectral modulation leads to a modulation of the amplitude of the detector signal. This has the advantage that the signal is transferred from a low frequency to a higher frequency, which separates the signal contribution used for hemoglobin detection and thus for PPG from motion artefacts, which typically occur at lower frequencies. The technique can also be described as creating a carrier and sidebands. The sidebands contain the message, which for PPG allows deriving the actual PPG signal, while the carrier does not. However, both carrier and sidebands are equally influenced by transmission channel variations, such as artefacts. Therefore, in one embodiment, the sideband signals, which contain the actual PPG signal, are corrected for channel variations (motion) using the amplitude of the carrier.

Furthermore, the employed technique enables AC coupling, freeing up a valuable dynamic range in the electronics used for the signal processing. Another advantage of the technique used is that the sensor principle is self-calibrating: The amplitude of the detected carrier signal received by the detector unit is a measure of the attenuation by the measured sample, while the modulation of the carrier amplitude is a measure for the desired hemoglobin detection or PPG information.

The described technique may also be used to detect species different from hemoglobin by their characteristic modulation frequency in accordance with the given absorption or transmittance characteristics, by suitably selecting the spectral modulation interval according to the described criteria and providing spectrally selective and tuneable transmittance information for electromagnetic radiation in the spectral modulation interval.

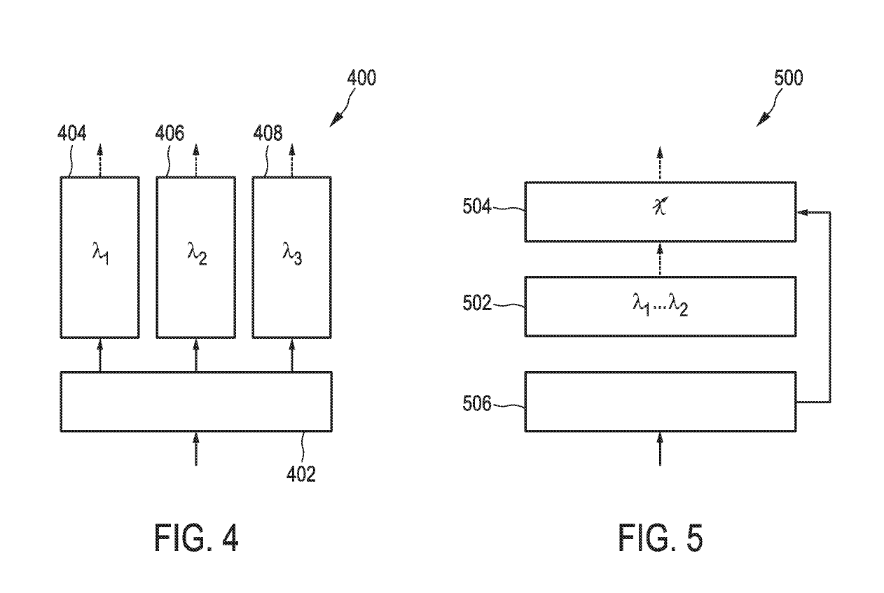

FIGS. 4 and 5 each show a schematic block diagram of emitter units 400 and 500 of different embodiments of a hemoglobin detection apparatus according to the present embodiment. The emitter units 400 and 500 are spectrally tuneable to selectively provide electromagnetic radiation at different selected wavelengths. They are therefore suitable for use as the emitter unit 106 of the hemoglobin detection apparatus 100 of FIG. 1.

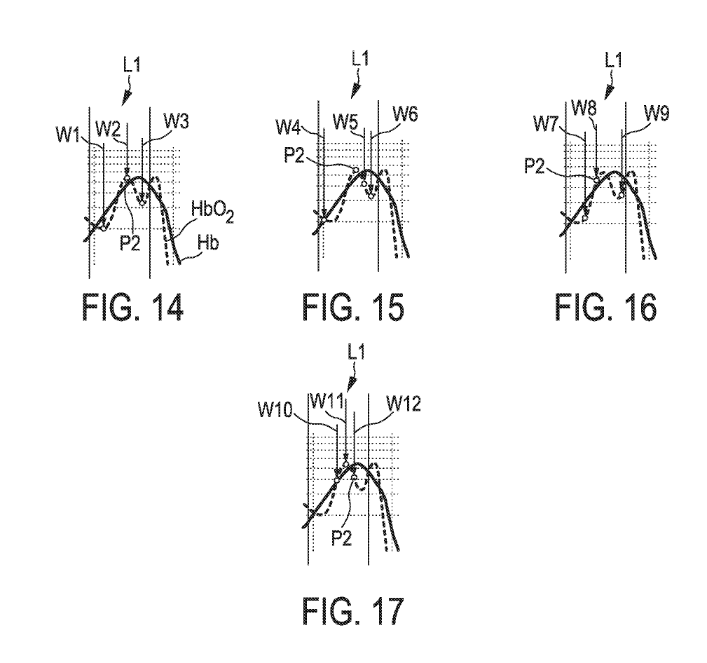

The emitter unit 400 of FIG. 4 comprises a driver unit 402 and three solid-state emitters 404, 406, and 408. Each of the solid-state emitters 404, 406, and 408 provides one fixed wavelength within the spectral modulation interval selected for use by the hemoglobin detection apparatus. The term “fixed wavelength” does not refer to a single wavelength, but to an emission spectrum with a peak wavelength and a suitable spectral width of emission. In one variant of the emitter unit 400, the spectral width of the solid-state emitters 404, 406, and 408 is small enough to allow determining transmittance information with each of the solid-state emitters in different sections of the selected spectral modulation interval 306. In one such variant, the solid-state emitters 404, 406, and 408 do not have any substantial spectral overlap of their emission spectra so that three discrete spectral regions within the selected spectral modulation interval 306 can be tested. As this example shows, there is no need to obtain the transmittance information within the selected spectral modulation interval 306 with high spectral resolution over the whole spectral modulation interval. It is sufficient to test certain sections or wavelengths of the spectral modulation interval so as to obtain sufficient signal contrast during a modulation period. Different alternative variants for wavelength selection will be described with reference to the FIGS. 14 to 18 further below.

Examples of suitable solid-state emitters 404 to 408 are light-emitting diodes, organic light-emitting diodes, and laser diodes. They can be provided as small devices allowing the provision of portable hemoglobin detection or PPG apparatus. These emitters are commercially available at any desired wavelength and with different spectral bandwidths, in particular in the visible spectral range.

The driver unit 402 receives the tuning control signal from the modulation control unit 108 (not shown in FIG. 4) in the form of a switching sequence that activates a respective one of the different solid-state light emitters 404, 406, and 408 at a respective phase of the modulation period.

Instead of three solid-state emitters, any other number of solid-state emitters can be used in the emitter unit 400. The selected number of solid-state emitters should be chosen suitably to obtain the necessary amount of the desired spectral transmittance information with a higher-frequency component substantially caused by hemoglobin only, in accordance with the functional description given above with reference to FIG. 3. The number of solid-state emitters with mutually non-overlapping emission spectra is preferably equal or larger than three, as will be discussed by way of different examples in the context of the description of FIGS. 14 to 18. As an alternative to using different solid-state emitters emitting electromagnetic radiation of different wavelengths, a corresponding number of identical broadband emitters may be used, each followed by a different spectral bandpass filter that allows transmission of only the respective selected wavelength.

In an alternative variant of the emitter unit 400, the solid-state light emitters 404 to 408 have different peak wavelengths, but overlapping emission spectra, which together cover a desired section of the selected spectral modulation interval. Mutually overlapping emission spectra can thus be used to provide a tuneable overall emission spectrum within the selected spectral modulation interval with a controllable peak wavelength as the respective weighted sum of the intensities of the individual solid-state emitters. In this variant, the the modulation control unit 108 is configured to provide the tuning control signal to the emitter unit 400 in the form of an intensity control signal that determines a respective relative intensity of each of the different solid-state emitters 404 to 408 at a respective phase of the modulation period. In this variant, the number of solid-state emitters with mutually overlapping emission spectra is equal or larger than three. A larger number of solid-state emitters allows achieving transmittance information with higher spectral resolution.

An alternative embodiment of an emitter unit 500 is shown in FIG. 5. In this embodiment, a single broad-band emitter 502 of electromagnetic radiation that spectrally covers the spectral modulation interval 306 between the wavelengths λ₁ and λ₂ is followed by a tuneable optical filter 504, which is configured to transmit the electromagnetic radiation at one of a plurality of different selectable wavelengths across the selected spectral modulation interval in dependence on the tuning control signal. The tuneable filter 506 is controlled by a driver unit 506. The driver unit receives the tuning control signal. This way, the modulation of the emitted wavelength is achieved by tuning the optical filter 506 as a spectral bandpass filter to sweep across different wavelengths of the spectral modulation interval under control of the modulation control unit 108.

In these and other embodiments, it is important that the spectral characteristics of the emitter-detector unit are taken into account to avoid detection errors. In particular, care should be taken that the intensity of the total emission provided by the emitter unit does not contain frequency components that form harmonics of the modulation frequency, possibly caused by the wavelength modulation itself Therefore, a feedback or feed forward (e.g. look-up-table approach) can be necessary. Assume for instance that four light-emitting diodes (LEDs) are used, where LED1 and LED3 emit a higher intensity than LED2 and LED4. The detected signal without appropriate correction of this intensity ratio will already contain even harmonics, whether there is hemoglobin seen or not. Therefore the intensities of the LEDs must be equalized in this situation. This can be done using a look-up table. It is also possible to measure the emitted light intensity 112 and use a feedback loop to equalize it. Another example is in the use of a tunable filter. These are never flat in their pass-band, and will therefore generate harmonics in the detected signal independent of the measurand.

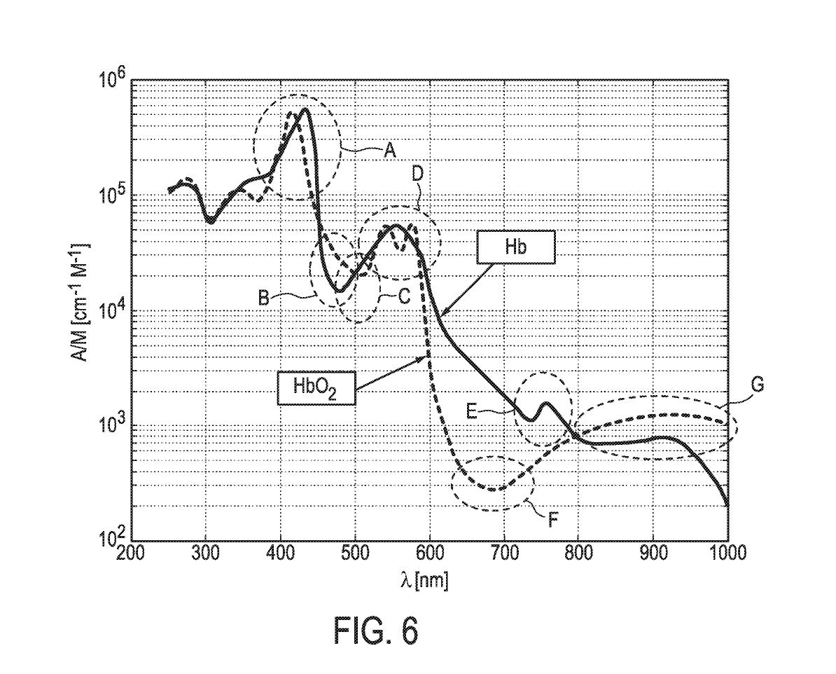

FIG. 6 is a diagram showing absorption properties of hemoglobin (Hb) and oxygenated hemoglobin (HbO₂) in a spectral range between 200 and 1000 nanometer. From FIG. 6 it can be seen that in the blue and green spectral ranges and also around 684 nm (red), parts of the absorption spectrum exhibit changes in the sign of the slope of the absorption spectra of hemoglobin. Suitable candidate features in the absorption spectra are marked by dashed ovals and labels A to G, and comprise maxima or minima of the respective absorption spectrum. Note that for normal healthy subjects, the hemoglobin absorption is almost fully determined by the oxygenated hemoglobin (HbO2) species due to the high oxygen saturation of arterial blood. However, the absorption peak F around 770 nanometer is unique for deoxygenated hemoglobin and may be used to specifically detect the presence of this species. The diagram of FIG. 6 thus allows identifying suitable candidate spectral ranges for implementation of embodiments of the hemoglobin detection apparatus of the present disclosure.

Whether or not a candidate spectral range is actually suitable for implementation must be determined by comparison of different absorption spectra in the candidate spectral ranges. Additional reference is therefore made to FIG. 7. FIG. 7 compares absorption properties of blood, shown under 700, with those of melanosome (702), epidermis (704) and skin (706), for use in identifying suitable spectral ranges A, B, C, D, and E, F and G for implementation of hemoglobin detection and PPG according the present disclosure. The preferred central wavelengths for suitable spectral ranges are approximately 418 nm, 512 nm, 542 nm, 560 nm, 576 nm and 684 nm, but other nonlinear parts in the spectrum are also possible for use in embodiments of the hemoglobin detection apparatus. This is so because any nonlinear transfer function will always generate higher harmonics. The main reason for choosing the above mentioned wavelengths is the change of slope that occurs there. This will give a direct frequency doubling while at other wavelength, third harmonics with lower amplitudes are more likely to be generated.

While in the suitable spectral ranges A to E changes in the sign of the slope of the absorption spectra of hemoglobin can be found, this is not the case in these spectral ranges for the shown interfering species.

In the following, different examples of suitable spectral modulation intervals based on the suitable spectral range C marked in FIG. 7 will be discussed with reference to FIGS. 8 to 13.

FIG. 8 is a section of the spectrum of FIG. 6. It illustrates a suitable spectral modulation interval L1 between spectral limits L11 and L12 in the spectral range C for use in an embodiment of the hemoglobin detection apparatus and of the PPG apparatus. In FIG. 8 and in FIGS. 9 to 13, spectral limits of the spectral modulation intervals L1 to L6 are indicated by full vertical lines. For further illustration of the working principle of an embodiment of the present embodiment, three spectral positions P1 to P3 are marked. Respective tangents S1 to S3 to the absorption spectrum of oxygenated hemoglobin at the spectral positions P1 to P3 are represented by dotted lines to indicate the respective slopes of the absorption spectrum at these spectral positions. As can be seen, the slope is positive at the spectral position P1, negative at the spectral position P3, and zero at the spectral position P2, at which the absorption spectrum forms a maximum. The spectral position is preferably selected with a spectral resolution that optimizes the detected signal intensity and also provides a good modulation contrast at the desired frequency component of the detector signal.

As the examples of different spectral modulation intervals L2 to L6 shown in FIGS. 9 to 13 show, different options exist for suitable spectral modulation intervals across the spectral absorption feature in the spectral region C. For graphical simplicity, the coordinate axes are omitted in FIGS. 9 to 13. They are identical to those of FIG. 8, as the same section of the absorption spectra of oxygenated and deoxygenated hemoglobin are shown. The shown spectral region contains two different resonance absorption peaks in the spectrum of oxygenated hemoglobin at the spectral positions P2 and P4, each of which can form a central wavelength of suitable spectral modulation intervals L2 and L3 shown in FIGS. 9 and 10. It is noted that the lower wavelength limit L3 includes the local minimum of spectral region C. This is allowed, but it will ‘leak’ energy to an odd harmonic and therefore will give sub-optimum modulation contrast. To obtain a better modulation contrast, the inclusion of the local minimum should be avoided.

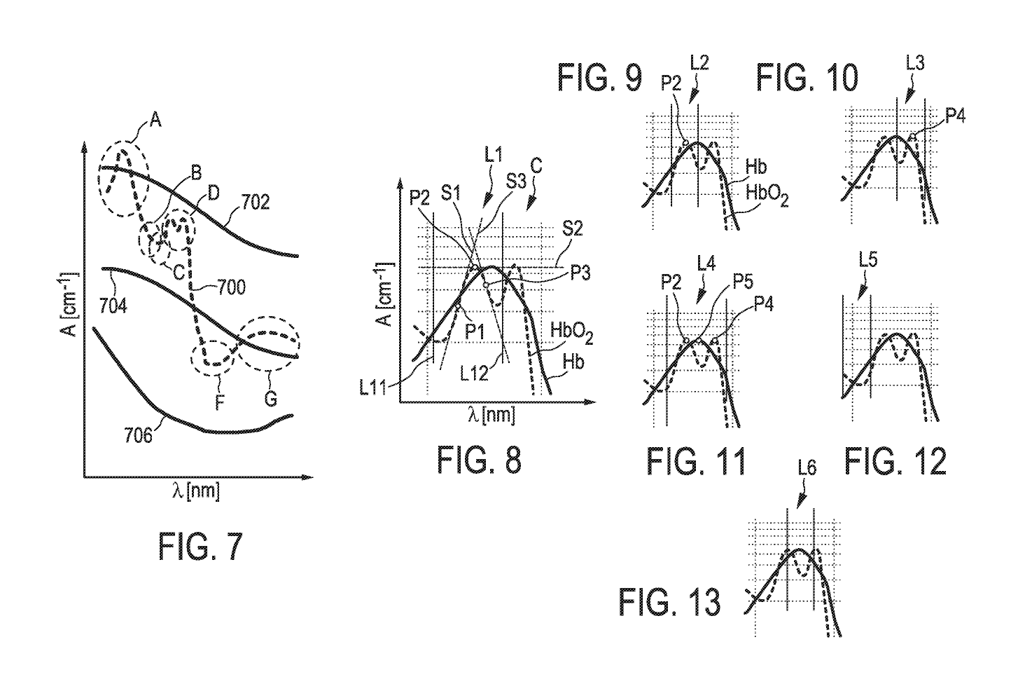

Another variant illustrated in FIG. 11 uses a spectral modulation interval L4 that covers both absorption peaks at P2 and P4. This allows achieving a desired signal contribution to the detector signal by oxygenated hemoglobin with a frequency component at the fourth harmonic of the modulation frequency. In the same spectral modulation interval L4, deoxygenated hemoglobin exhibits a single absorption peak at a spectral position P5, which generates a signal contribution with a frequency component at the second harmonic of the modulation frequency. This allows obtaining clearly separable signal contributions by oxygenated and deoxygenated hemoglobin to the detector signal. The two different frequency components of the detector signal generated by oxygenated and deoxygenated hemoglobin can be separated by frequency filtering and used to determine an estimate of a relative amount of oxygenated an deoxygenated hemoglobin, which is indicative of an amount of oxygen saturation of the blood. FIGS. 12 and 13 show two further variants of spectral modulation intervals L5 and L6, which comprise a respective minimum of the absorption of oxygenated hemoglobin. Such minima are equally suitable as maxima for generating a second-harmonic contribution to the detector signal. Whereas the minimum in the spectral modulation interval L5 is exclusive to oxygenated hemoglobin and allows detecting this species alone, the spectral modulation interval comprises the minimum between the absorption peaks of oxygenated hemoglobin at the spectral positions P2 and P4 as well as the absorption maximum of deoxygenated hemoglobin at the spectral position P5. Depending on the oxygen saturation, the modulation contrast at the second harmonic will be larger or lower in this variant. This can also be used for obtaining a measure for oxygen saturation.

FIGS. 14 to 17 illustrate further variants of hemoglobin detection apparatus and PPG apparatus. More specifically, FIGS. 14 to 17 show different alternative sets of suitable wavelengths for an implementation of different embodiments of a hemoglobin detection or PPG apparatus based on the same spectral modulation interval L1 of FIG. 8. As a guideline, the wavelengths or, in other words, spectral positions for testing the transmittance of hemoglobin and other species should be chosen to lie on different sides of the absorption feature that has been selected for hemoglobin detection by choosing the given spectral modulation interval L1.

It is pointed out that it is not a requirement that the set of wavelengths selected for testing the transmittance corresponds to spectral positions at which the transmittance as a function of wavelength exhibits slopes of opposite signs. As an example, the wavelengths W1 to W3 used in a first variant shown in FIG. 14 are at spectral positions across the absorption maximum at the spectral position P2, at which the absorption (and thus also the transmittance) exhibits a substantially identical slope of zero or close to zero. For these spectral positions all correspond to extrema in the absorption spectrum of oxygenated hemoglobin. The modulation contrast at the second harmonic of the modulation frequency that is achieved with the selection of the wavelengths W1 to W3 is rather high, which is advantageous.

As a second variant, FIG. 15 shows a set of testing wavelengths W4 to W6 at three other spectral positions on both sides of the absorption peak at the spectral position P2. For all three testing wavelengths W3 to W6, the slope in the absorption spectrum of oxygenated hemoglobin is negative. The modulation contrast at the second harmonic is somewhat lower than in the variant of FIG. 14.

A third set of testing wavelengths W7 to W9 shown in FIG. 16 includes three spectral positions where the slope in the absorption spectrum of oxygenated hemoglobin is positive. The modulation contrast at the second harmonic is similar to that of the variant of FIG. 14.

A fourth set of testing wavelengths W10 to W12 is selected to be at spectral positions closely spaced around the absorption peak at the spectral position P2. The achieved modulation contrast is rather low in this example.

The modulation contrast achieved in any of the variants described also depends on the spectral resolution of the transmittance measurement. A lower spectral resolution can be used while still achieving a high modulation contrast, if the spectral distance between the testing wavelengths is larger and if the spectral line width of the absorption feature of hemoglobin used for the measurement allows.

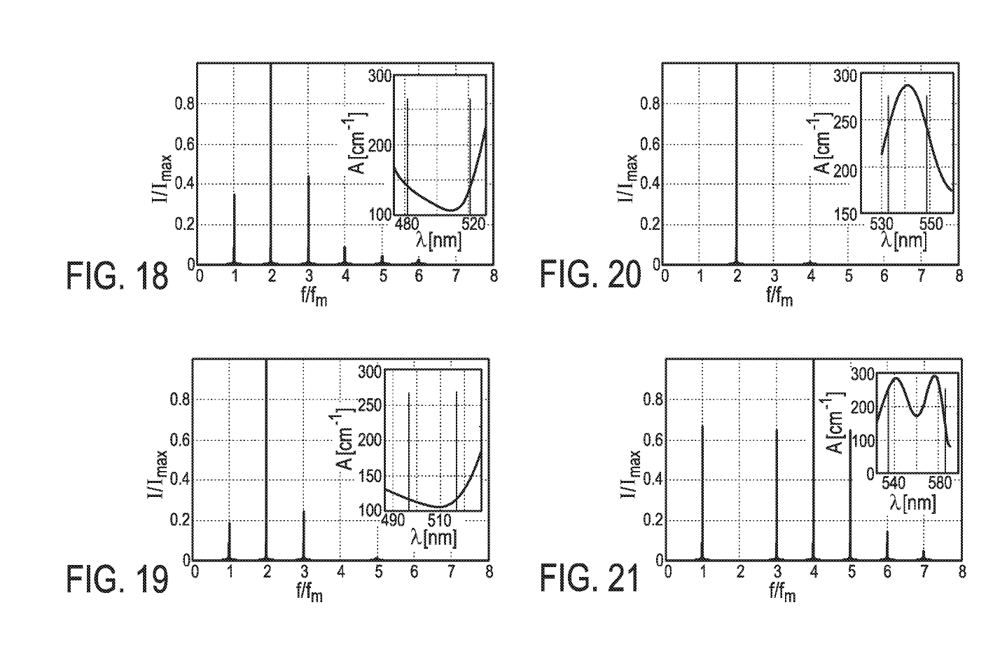

FIGS. 18 to 21 show examples of a frequency distribution of the detector signal for different spectral modulation intervals in the implementation of different embodiments of a hemoglobin detection apparatus.

Each of the FIGS. 18 to 21 has a main diagram and an inserted diagram. The respective inserted diagrams are plots of respective sections of the absorption spectrum of oxygenated hemoglobin shown in FIG. 6 under HbO₂, to illustrate a respective spectral modulation interval selected for testing the transmittance of (and thus detecting) oxygenated hemoglobin. The respective limits of the spectral modulation intervals are indicated by full vertical lines reaching down to the wavelength scale at the abscissa of the inserted diagram.

The respective main diagrams of FIGS. 18 to 21 show a relative amplitude I/Iₘₐₓ of different harmonic frequency components that form contributions to the detector signal, plotted as a function of the ratio of frequency f to the modulation frequency fₘ. In other words, the main diagrams illustrate a Fourier spectrum showing the relative amplitude of the different harmonics of the modulation frequency in the respective detector signal obtained when measuring in the respective selected spectral modulation interval. The results shown in the main diagrams are derived from a simulation based on the respective absorption spectra shown and based on a continuous sinusoidal wavelength modulation between the respective limits of the spectral modulation interval at a modulation frequency of 500 Hz. The examples shown are non-limiting with respect to any of their underlying parameters.

The spectral modulation interval underlying FIG. 18 has a central wavelength of 506.8 nm and a spectral width of 20 nm between its spectral limits. This spectral modulation interval gives rise to a dominating contribution of the second harmonic of the modulation frequency fₘ, having an amplitude that more than doubles the amplitude of the third harmonic, followed by even weaker contributions of the first and forth harmonics, and very weak contributions of the fifth and sixth harmonics.

For comparison, an only slight modification of the spectral modulation interval forms the basis of the detector signal represented by FIG. 19. The spectral modulation interval used here has the same central wavelength of 506.8 nm, but a reduced spectral width of only 10 nm. Otherwise, the same parameters are employed. With this modification, the relative amplitudes of the frequency components other than the second harmonic are strongly reduced in comparison with the example of FIG. 18, such that only the third and first harmonic remain visible in the Fourier spectrum, with relative contributions of less than 0.3 and less than 0.2, respectively. Thus, an optimization of the desired output signal can be achieved by this modification of the spectral modulation interval.

The spectral modulation interval underlying the example of FIG. 20 has a central wavelength of 540.9 nm and a spectral width of 8 nm between its spectral limits. This spectral modulation interval gives rise to a contribution of oxygenated hemoglobin to the detector signal that is determined substantially alone by the second harmonic of the modulation frequency fₘ. This spectral modulation interval thus achieves a very advantageous output signal. This is due to the fact that in this spectral modulation interval the absorption spectrum of oxygenated hemoglobin can almost perfectly fitted by, i.e., decomposed into a parabolic function.

The spectral modulation interval underlying the example of FIG. 21 has a central wavelength of 560 nm and a spectral width of 26 nm between its spectral limits. This spectral modulation interval gives rise to a contribution of oxygenated hemoglobin to the detector signal that is has a dominant component at the fourth harmonic of the modulation frequency fₘ, but substantially no contribution by the second harmonic. As FIG. 6 shows, deoxygenated hemoglobin (Hb) has an absorption in this spectral modulation interval that has decomposition with a strong contribution of a parabolic function. Thus, the presence of a second harmonic in the detector signal of this embodiment will be an indication of the presence of deoxygenated hemoglobin. This spectral modulation interval can thus be used for distinguishing between deoxygenated hemoglobin (second harmonic) and oxygenated hemoglobin (fourth harmonic) and, by evaluating the respective amplitudes in the frequency domain, therefore allows determining an estimate of the peripheral capillary oxygen saturation (also referred to as SpO₂).

The following description turns to embodiments of a hemoglobin detection method and of a PPG method in accordance with embodiments of the present disclosure.

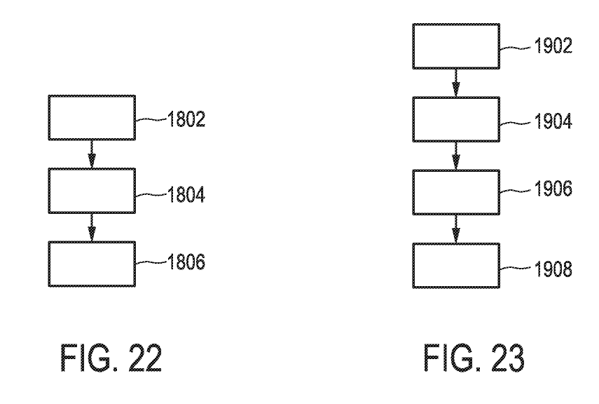

FIG. 22 is a flow diagram showing an embodiment of a hemoglobin detection method. The method is based on using a spectrally tuneable emitter-detector unit and comprises a step 1802 of providing a periodic spectrally selective emission and detection of electromagnetic radiation at different wavelengths that cover a spectral modulation interval during a respective modulation period. The spectral modulation interval may for instance be selected according to a criterion that a quantity indicative of a transmittance of hemoglobin, plotted as a function of wavelength, exhibits a nonlinear spectral dependence in the spectral modulation interval that can be decomposed with a significant contribution by at least one even function, and that in the spectral modulation interval the quantity indicative of the respective transmittance of other species to be exposed to the electromagnetic radiation emitted and detected, plotted as a function of wavelength, do not exhibit a nonlinear spectral dependence that can be decomposed with a significant contribution of at least one even function.

A subsequent step 1804 comprises providing a detector signal indicative of the detected electromagnetic radiation as a function of time.

A further step 1806 comprises processing the detector signal and providing an output signal, which is indicative of a contribution of at least one frequency component that forms a second or higher harmonic of the modulation frequency to the detector signal.

Variants of this embodiment correspond to the variants of the hemoglobin detection apparatus described hereinabove.

FIG. 23 is a flow diagram showing an embodiment of a PPG method. The photoplethysmography method is based on performing a hemoglobin detection method according to the embodiment of FIG. 22. Therefore, the steps 1902 to 1906 are identical to the steps 1802 to 1806, respectively. The method further comprises a step 1908 of determining cardiovascular status information from the output signal and providing the cardiovascular status information.

Variants of this embodiment correspond to the variants of the hemoglobin detection apparatus and PPG apparatus described hereinabove.

In summary, a hemoglobin detection apparatus comprises a spectrally tuneable emitter-detector unit, which is configured to emit or detect electromagnetic radiation spectrally selectively and periodically at different wavelengths covering a spectral modulation interval at a modulation frequency, and to provide a detector signal indicative of the detected electromagnetic radiation as a function of time. The apparatus further comprises a signal processing unit, which is configured to receive the detector signal and to provide an output signal, which is indicative of a contribution of at least one frequency component that forms a second or higher even harmonic of the modulation frequency to the detector signal. The hemoglobin detection apparatus can be used in photoplethysmography applications.

In accordance with embodiments of the present disclosure, hemoglobin detection is achieved by spectrally modulated emission or detection of the transmittance of hemoglobin and other species exposed to a modulated wavelength, and by phase or frequency specific detection means such as band-pass filtering or a synchronous detector or lock-in amplifier. Any nonlinear transfer function that generates even harmonics and may serve as a basis for selective hemoglobin detection if other species exposed to the electromagnetic radiation do not contain an equally strong nonlinear transfer function generating even harmonics in the selected spectral modulation interval.

While the embodiment has been illustrated and described in detail in the drawings and foregoing description, such illustration and description are to be considered illustrative or exemplary and not restrictive; the embodiment is not limited to the disclosed embodiments. Other variations to the disclosed embodiments can be understood and effected by those skilled in the art in practicing the claimed embodiment, from a study of the drawings, the disclosure, and the appended claims.

In the claims, the word “comprising” does not exclude other elements or steps, and the indefinite article “a” or “an” does not exclude a plurality.

A single stage or other unit may fulfill the functions of several items recited in the claims. The mere fact that certain measures are recited in mutually different dependent claims does not indicate that a combination of these measured cannot be used to advantage.

Any reference signs in the claims should not be construed as limiting the scope.

1. A hemoglobin detection apparatus, comprising
a spectrally tuneable emitter-detector unit, which is configured to emit or detect electromagnetic radiation spectrally selectively and periodically at different wavelengths covering a spectral modulation interval (L1 to L6) at a modulation frequency (fₘ), and to provide a detector signal indicative of the detected electromagnetic radiation as a function of time; and
a signal processing unit, which is configured to receive the detector signal and to provide an output signal, which is indicative of a contribution of at least one frequency component that forms a second or higher even harmonic of the modulation frequency to the detector signal.

2. The hemoglobin detection apparatus of claim 1, wherein the spectral modulation interval is at least one from a group of spectral intervals (A to G) in which a quantity indicative of a transmittance of hemoglobin (Hb, HbO₂), plotted as a function of wavelength, exhibits a nonlinear spectral dependence that can be decomposed with a significant contribution by at least one even function, and in which spectral modulation interval the quantity indicative of the respective transmittance of other species to be exposed to the electromagnetic radiation emitted and detected, plotted as a function of wavelength, does not exhibit a nonlinear spectral dependence that can be decomposed with a significant contribution of at least one even function.

3. The hemoglobin detection apparatus of claim 1, wherein the spectral modulation interval comprises a wavelength (P2), at which oxygenated hemoglobin exhibits a local peak (A, C, E) or a local minimum (B, D) of absorbance.

4. The hemoglobin detection apparatus of claim 1, wherein the signal processing unit is configured to provide as the output signal a contribution of the second harmonic of the modulation frequency to the detector signal.

5. The hemoglobin detection apparatus of claim 1, further comprising a modulation control unit, which is configured to provide a tuning control signal, which is periodic at a modulation frequency (fₘ) for driving a spectrally modulated emission or detection of electromagnetic radiation by the emitter-detector unit that covers the spectral modulation interval during a respective modulation period (T).

6. The hemoglobin detection apparatus of claim 5, further comprising a spectral alignment unit, which is configured
to control the modulation control unit in performing a spectral alignment process by testing different candidate wavelengths (W1 to W12) in a spectral modulation interval (L1) around a given central wavelength;
to determine from the respective detector signals received for the different candidate wavelengths an optimal spectral modulation interval (L1 to L6), at which the contribution of the second or higher even harmonic of the modulation frequency to the detector signal is relatively the largest; and which is configured
to select the optimal spectral modulation interval as the spectral modulation interval to be used for regular hemoglobin detection operation by the modulation control unit.

7. The hemoglobin detection apparatus of claim 5, wherein the emitter-detector unit comprises
a spectrally tuneable emitter unit, which is configured to selectively provide the electromagnetic radiation at different wavelengths in dependence on the tuning control signal; and
a detector unit, which is configured to provide a detector signal that is indicative of an amount of electromagnetic radiation emitted by the emitter unit and scattered by blood and other species of a subject, as a function of time.

8. The hemoglobin detection apparatus of claim 5, wherein the signal processing unit comprises a lock-in amplifier, which receives the tuning control signal and the detector signal.

9. The hemoglobin detection apparatus of claim 1, wherein the emitter unit, comprises at least one tuneable solid-state emitter.

10. The hemoglobin detection apparatus of claim 1, wherein the emitter unit comprises a plurality of different solid-state emitters, each providing one fixed wavelength within the spectral modulation interval, and to activate a respective one of the different solid-state light emitters at a respective phase of the modulation period.

11. The hemoglobin detection apparatus of claim 1, wherein the emitter-detector unit comprises a spectrally tuneable detector unit, which is configured to selectively detect the electromagnetic radiation at different wavelengths and to provide a detector signal that is indicative of an amount of the spectrally selected electromagnetic radiation emitted by the emitter unit and scattered by blood and other species of a subject, as a function of time.

12. The hemoglobin detection apparatus of claim 7, wherein either the emitter unit or the detector unit comprises a tuneable optical filter, which is configured to transmit the electromagnetic radiation at one of a plurality of different selectable wavelengths across the spectral modulation interval.

13. A photoplethysmography apparatus, comprising a hemoglobin detection apparatus according to claim 1 and a PPG evaluation unit, which receives the output signal and is configured to determine cardiovascular status information from the output signal and provide the cardiovascular status information.

14. A hemoglobin detection method, comprising
periodically providing, at a modulation frequency, a spectrally selective emission and detection of electromagnetic radiation at different wavelengths that during a respective modulation period cover a spectral modulation interval (L1 to L6);
providing a detector signal indicative of the detected electromagnetic radiation as a function of time; and
processing the detector signal and providing an output signal, which is indicative of a contribution of at least one frequency component that forms a second or higher even harmonic of the modulation frequency to the detector signal.

15. A photoplethysmography method, comprising a hemoglobin detection method according to claim 14 and further comprising determining cardiovascular status information from the output signal and providing the cardiovascular status information.
